(19) United States
(12) Patent Application Publication
(10) Pub. No.: US 2014/0066595 A1
(43) Pub. Date: Mar. 6, 2014

(54) Modulators of Protein Production in a Human Cell Line and Cell-free Extracts Produced Therefrom

(71) Applicants: Emily Anderson, Arvada, CO (US); Peter Bell, South Beloit, IL (US); Penny Jensen, Marengo, IL (US); Jon Karpilow, Boulder, CO (US); Anja Smith, Thornton, CO (US); Krishna Vattem, Roscoe, IL (US); Brian Webb, Roscoe, IL (US); Devin Leake, Denver, CO (US); Alex Medford, Firestone, CO (US); Elena Maksimova, Louisville, CO (US); Craig Smith, Oregon, WI (US)

(72) Inventors: Emily Anderson, Arvada, CO (US); Peter Bell, South Beloit, IL (US); Penny Jensen, Marengo, IL (US); Jon Karpilow, Boulder, CO (US); Anja Smith, Thornton, CO (US); Krishna Vattem, Roscoe, IL (US); Brian Webb, Roscoe, IL (US); Devin Leake, Denver, CO (US); Alex Medford, Firestone, CO (US); Elena Maksimova, Louisville, CO (US); Craig Smith, Oregon, WI (US)

(73) Assignee: THERMO FISHER SCIENTIFIC BIOSCIENCES INC., Lafayette, CO (US)

(21) Appl. No.: 14/010,246

(22) Filed: Aug. 26, 2013

Related U.S. Application Data
(60) Provisional application No. 61/696,404, filed on Sep. 4, 2012.

(51) Int. Cl. C12P 21/00; C07K 14/00

(52) U.S. Cl. CPC C12P 21/00; C07K 14/00
USPC 530/350; 435/69.1

(57) ABSTRACT

Cell-free extracts, methods for producing these extracts, methods for using these extracts, compositions that facilitate production of these extracts and kits that contain these extracts are provided. By increasing or decreasing certain gene products through, for example, the use of siRNA or mimics, one can develop mammalian cell-free extracts that have desired levels of efficiency.

### CROSS-REFERENCE TO RELATED APPLICATION

This application claims the benefit of the filing date of U.S. Provisional Application Ser. No. 61/696,404, filed on Sep. 4, 2012, the entire disclosure of which is incorporated by reference as if set forth fully herein.

### FIELD OF INVENTION

The present invention relates to cell-free extracts and cell-free protein expression.

### BACKGROUND OF THE INVENTION

In vitro protein expression is a technique that enables researchers to express and to manufacture small amounts of functional proteins. Among its advantages over in vivo techniques are the convenience and the reduced time that in vitro expression affords. When using in vitro expression systems, one can produce proteins rapidly because in these systems there are no requirements of gene transfection, cell culture maintenance or extensive protein purification.

Current widely used in vitro protein translation systems include those isolated from bacteria, yeast, drosophila, wheat germ and rabbit reticulocytes. To a lesser degree, cell-free protein synthesis systems that are derived from cultured mammalian cells are also currently being used. These systems are of particular interest because they offer the possibility of modifying proteins after translation. Being able to cause post-translational modification is important for the analysis of gene products in eukaryotes and for research in connection with translational regulation.

The Thermo In Vitro Protein Expression System (IVPE) is one example of a cell-free translation system that is created from a human cell line and that allows for expression of mammalian proteins in a mammalian system. The IVPE system has been shown to generate a higher protein output than that of the well-known rabbit reticulocyte lysate system, and as with most cultured human cell lines, there is smaller batch-to-batch variability as compared to that of rabbit reticulocyte lysate systems. Thus, the cells of this cultured cell line are more uniform than those of rabbit blood. However, despite the benefits of the IVPE system, there is always a desire to improve protein yields further.

### SUMMARY OF THE INVENTION

The present invention provides cell-free extracts that demonstrate efficient translational capabilities. The present invention also provides molecules and methods for producing these extracts, as well as methods for using these extracts.

According to a first embodiment, the present invention provides a cell-free extract comprising a product of a gene selected from the group consisting of: CCL19, GPR62, LILRB1, MAP3K14, MRPL14, OPN5, SCNN1A, SLC37A2 and TTYH3.

According to a second embodiment, the present invention provides an extract prepared from cells in which expression of a gene is inhibited, wherein the gene is selected from the group consisting of: CCL19, GPR62, LILRB1, MAP3K14, MRPL14, OPN5, SCNN1A, SLC37A2 and TTYH3. In some embodiments, expression of one or two or more of these genes is inhibited. By way of non-limiting examples, the inhibition may cause reduced expression of the inhibited gene or when expression of a plurality of genes is inhibited, reduced expression of each of the genes. In some embodiments, expression of the protein or proteins that are typically generated from these genes may be completely silenced or the protein or proteins may be expressed at a level of less than 80%, less than 70%, less than 60%, less than 50%, less than 40%, less than 30%, less than 20% or less than 10% of the uninhibited gene or genes.

According to a third embodiment, the present invention provides a method of making a cell-free extract comprising: (a) establishing a stable cell line harboring at least one shRNA construct capable of expressing a double stranded oligonucleotide, wherein the double stranded oligonucleotide inhibits expression of a gene selected from the group consisting of: CCL19, GPR62, LILRB1, MAP3K14, MRPL14, OPN5, SCNN1A, SLC37A2 and TTYH3; and (b) collecting extract from the cell. In this embodiment, the shRNA may contain and/or code for an antisense sequence that is at least 80%, at least 90% or 100% complementary to a coding or non-coding region of the mRNA that corresponds to one or more of the aforementioned genes. The shRNA may also code for a sense sequence that is at least 80%, at least 90% or 100% complementary to the antisense sequence. Molecules generated from an shRNA may cause partial or complete gene silencing by making use of cellular RNAi machinery to inhibit expression of a protein encoded by a gene.

The double stranded RNA oligonucleotide that is produced may itself be a single-stranded polynucleotide that has a stem-loop structure with one or more regions of complementarity to a target and to another one or more regions within the single-stranded polynucleotide, or it may start as an shRNA that is cleaved into two separate strands that have one or more regions of complementarity to each other. Alternatively, rather than starting with a single shRNA, one may start with two separate constructs, each of which has at least one region of at least 80%, at least 90% or 100% complementarity to a region of the other construct and one of the strands has a region of at least 80%, at least 90% or 100% complementarity to the target.

According to a fourth embodiment, the present invention provides a method of making a cell-free extract comprising: (a) establishing a stable cell line harboring at least one shRNA construct capable of expressing a double stranded oligonucleotide, wherein the double stranded oligonucleotide comprises and/or codes for a sequence that is complementary to a region of a target gene selected from the group consisting of: CCL19, GPR62, LILRB1, MAP3K14, MRPL14, OPN5, SCNN1A, SLC37A2 and TTYH3; and (b) collecting extract from the cell. Optionally, the region of the shRNA that is complementary to the target is also complementary to another region of the same shRNA.

In both the third and the fourth embodiments, the double stranded oligonucleotide may directly cause gene silencing through the use of a cell's RNAi machinery. However, expression of a protein may additionally or alternatively be affected indirectly due to off-target effects or intended targeting of an RNA sequence that indirectly controls expression of a protein of interest. Thus, expression of a target gene, which may be referred to as a first gene, may be increased or suppressed by the double stranded polynucleotide acting on second gene. For example, partial or complete inhibition of the second gene may cause partial or complete inhibition of the first gene, because the second gene regulates the first gene and/or they are part of the same pathway. In other embodiments, partial or complete inhibition of a second gene may cause increased expression of a first gene. By way of non-limiting examples, either one or both of the first and second genes may be selected from the group consisting of: CCL19, GPR62, LILRB1, MAP3K14, MRPL14, OPN5, SCNN1A, SLC37A2 and TTYH3.

According to a fifth embodiment, the present invention provides a method of making a cell-free extract comprising introducing at least one miRNA mimic selected from the group consisting of mimics of hsa-miR-155, hsa-miR-1912, hsa-miR-200b, hsa-miR-200c, hsa-miR-219-2-3p, hsa-miR-299-3p, hsa-miR-451, hsa-miR-634, hsa-miR-877*, and hsa-miR-941 into a cell. The mimic may be introduced passively or actively. Additionally or alternatively, one may introduce a vector that generates the mimic in the cell. Optionally, the cell may be incubated and then an extract may be collected.

According to a sixth embodiment, the present invention is directed to miRNA mimics. These compositions may be formed from oligonucleotides that are custom designed and that consist of, consist essentially of or comprise one or more sequences such as those that appear in Table II or Table III and/or a complement of any of those sequences. These oligonucleotides may be isolated, and they may exist in a purified or unpurified state. Additionally, they may be part of, or associated with, a vector, a cell or a kit. As persons of ordinary skill in the art will recognize, the oligonucleotides may be introduced directly into a cell. Alternatively, a vector that is capable of generating them may first be introduced into the cell. Optionally, the cell may be incubated, and then an extract may be collected. In some embodiments, the mimic is distinct from naturally occurring miRNA molecules. In those embodiments the mimic may differ from the naturally occurring sequence by the addition, deletion or substitution of one or more nucleotides.

According to a seventh embodiment, the present invention is directed to an siRNA, an miRNA mimic, a plurality of the aforementioned compositions, a combination of the aforementioned compositions, or kits comprising one or more of the aforementioned compositions or combinations thereof. Examples of these compositions include but are not limited to oligonucleotides that comprise, consist essentially of, or consist of one or more sequences disclosed in one or more of the tables of the present patent application and/or a complement to one or more those sequences. As persons of ordinary skill in the art will appreciate, when a target is referenced in the table, the molecule that will act upon it, will preferably comprise, consists essentially of or consist of the complement to the target. When a mimic is referenced in the table, the molecule that will be active in the cell or extract will preferably comprise, consists essentially of or consist of the referenced sequence. However, if the molecule that is introduced is to be used as a template from which to make the molecule that will act upon the target, then the molecule to be introduced will preferably comprise, consists essentially of or consist of the same sequence as the target or the complement of the mimic. In some embodiments, the molecule that is introduced will be double stranded and comprise, consists essentially of or consist of a sequence that is at least 80%, at least 90% or 100% complementary to the sequence referenced in one of the tables and comprise, consists essentially of or consist of a sequence that is at least 80%, at least 90% or 100% the same as the sequence referenced in one of the tables.

According to an eighth embodiment, the present invention provides a method of making a cell-free extract comprising: (a) introducing an oligonucleotide into a cell, wherein the oligonucleotide reduces expression of a gene and wherein a product of the gene negatively regulates translation; and (b) collecting an extract from the cell. Thus, introduction of the oligonucleotide will increase translation.

According to a ninth embodiment, the present invention provides a method of making a cell-free extract comprising introducing at least one oligonucleotide comprising, consisting essentially of or consisting of the complement of at least one of SEQ ID NO: 1-36 into a cell and collecting an extract from the cell.

According to a tenth embodiment, the present invention provides a method of making a cell-free extract comprising introducing at least one oligonucleotide comprising, consisting essentially of or consisting of the complement of at least one of SEQ ID NO: 37-48 into a cell and collecting an extract from the cell.

According to a eleventh embodiment, the present invention provides a method of making a cell-free extract comprising introducing at least one oligonucleotide comprising, consisting essentially of or consisting of at least one of SEQ ID No: 49-58 into a cell and collecting an extract from the cell.

According to an twelfth embodiment, the present invention comprises a method for creating a cell-free extract comprising introducing at least one oligonucleotide comprising, consisting essentially of or consisting of the complement of sequence of one of SEQ ID NO: 37-48 into a cell; and introducing at least one oligonucleotide comprising, consisting essentially of or consisting of the sequence of one of SEQ ID NO: 49-58 into the cell and collecting extract from the cell. The oligonucleotides may be introduced simultaneously or sequentially.

Collectively, the siRNA, shRNA and miRNA described herein may be referred to as RNAi reagents. Through the use of various of these RNAi reagents individually or in combination, one may develop cell lines and/or cell-free extracts that allow for efficient and effective modulation of protein synthesis.

### BRIEF DESCRIPTION OF THE FIGURES

FIG. 1 is a representation of Gluc (Gaussia Princeps Luciferase) expression and viability of unstressed cells in the presence of high doses of amiloride normalized to no amiloride at 18 hours.

FIG. 2 is a representation of cell viability under ER (endoplasmic reticulum) stress normalized to no stress at 18 hours.

FIG. 3 is a representation of Gluc expression under ER stress in the presence of amiloride normalized at 18 hours.

### DETAILED DESCRIPTION

Various embodiments of the present invention are directed to molecules, cell-free extracts, methods of making cell-free extracts and methods of using cell-free extracts. The cell-free extracts may impart increased efficiency when translating messenger RNA (mRNA) into proteins.

According to a first embodiment, the present invention provides a cell-free extract comprising a product of a gene selected from the group consisting of: CCL19, GPR62, LILRB1, MAP3K14, MRPL14, OPN5, SCNN1A, SLC37A2 and TTYH3. Preferably, the amount of the product of the gene is present in an amount that is less than 40%, less than 30%, less than 20% or less than 10% of the native level. For example, in some embodiments, the amount of the protein that is present is between 5% and 40% or between 10% and 30% of the native level.

For each of these genes, Table I provides the gene name, Entrez Gene ID and target accession number. The sequence of each gene may be obtained through the target accession number at the NCBI database, which is incorporated by reference.

A “cell-free extract” refers to an extract from a cell that comprises cytosolic and organelle components. A cell-free extract may also be referred to as a “cell-free lysate.” In some embodiments, no components or only de minimus components from the nucleus of a cell are present in the cell-free lysate.

The product of the gene may comprise, consist essentially of or consist of one or more of RNA, DNA, a DNA/RNA hybrid, an oligonucleotide, a poly-amino acid sequence or a protein that may be determined in whole or in part based on the gene sequence referenced in Table I and the transcription and translation of the gene. In some embodiments, products are present from two, three, four, five, six, seven or eight of the genes identified in Table I. These products may have been generated by the cell and thus be part of the extract that has been collected and/or the products may be added to the extract after having been produced elsewhere, e.g., by chemical or enzymatic synthesis or a combination thereof. Depending on the desired concentration of the product or products, a person of ordinary skill in the art may suppress or enhance production of the product (e.g., protein) or products (e.g., proteins) prior to forming the extract or after forming the extract.

The product of the gene may be the product of the naturally occurring gene or it may be a product of a gene in which one or more mutations, deletions or substitutions are present relative to the naturally occurring gene. In some embodiments, any product that is produced from a gene that has a mutation, deletion or substitution is as active as or more active than the corresponding naturally occurring product. In any embodiment in which the product that is produced from a non-naturally occurring gene is less active than the naturally occurring product, preferably the non-naturally occurring product is at least 50%, at least 60%, at least 70%, at least 80% or at least 90% as active as the naturally occurring product. In some embodiments, any mutation, deletion or substitution is within an active site, whereas in other embodiments, it is not within an active site. Examples of the locations of certain mutations, deletions and substitutions of nucleotides and their effects on products are reported in literature and may be accessed through publically available databases. For example, the National Center of for Biotechnology of the United States of America's National Institute of Health allows persons to look for all SNPs associated with a gene. See e.g., the instructions for searching for SNPs, which are located on the world-wide web at ncbi.nlm.nih.gov/guide/howto/view-all-snps/, and the database, which is accessible on the world-wide web at ncbi.nlm.nih.gov/gene. Both web-pages and the information accessible through them are incorporated by reference.

When the extract is being used for protein expression, preferably it also contains both the genetic template (mRNA or other RNA) that encodes the protein to be expressed, and a reaction solution that contains the necessary translational molecular machinery and any transcriptional molecular machinery that is required (if the template is e.g., DNA) in order to generate the desired molecule from the template. Additionally, preferably one or more, if not all, of the following components are present: (i) RNA polymerase for mRNA transcription; (ii) ribosomes for polypeptide translation; (iii) tRNA and amino acids; (iv) enzymatic cofactors and an energy source; and (v) cellular components that facilitate proper protein folding. When any components are present, preferably they are present in an amount and a concentration that are effective to carry out their intended purposes. As persons of ordinary skill in the art are aware, cell lysates can provide the correct composition and proportion of enzymes and building blocks that are required for translation. However, often in order to sustain synthesis one may add an energy source and amino acids. Further, optionally one may supplement the amount of any of the aforementioned components.

Preferably one removes the plasma membranes when obtaining an extract, thereby leaving only cytosolic and organelle components of the cell.

The cell-free extract may be part of a kit that also comprises mRNA for translation. Within a kit, mRNA may be kept in a separate container from the extract, and when a person of ordinary skill in the art wishes to start translation, he or she may combine the contents of the compartments under conditions conducive for translation. Alternatively, the kit does not contain the mRNA, and the user supplies the mRNA from a different source.

The products of the aforementioned genes may have one or more functions, including but not limited to inhibiting transcription of a nucleotide sequence, inhibiting translation of a nucleotide sequence, inhibiting transport of a protein or a polynucleotide, and inhibiting secretion of a protein or a polynucleotide.

According to a second embodiment, the present invention is directed to an extract prepared from cells in which expression of a gene is inhibited wherein the gene is selected from the group consisting of: CCL19, GPR62, LILRB1, MAP3K14, MRPL14, OPN5, SCNN1A, SLC37A2 and TTYH3. In some embodiments, inhibition is by at least 60%, at least 70%, at least 80%, at least 90% or 100%. In one non-limiting example, inhibition is by between 80% and 90%.

By way of a non-limiting example, the expression of the gene in the cell may be inhibited by RNA interference through the use of siRNA technology. siRNA technology refers to the use of short interfering ribonucleic acids that typically are formed either by: (1) two separate strands of oligonucleotides that form a duplex region that is 18-30 base pairs in length, and that each independently has no overhang regions or has one or more 5′ or 3′ overhang regions that are one to six nucleotides in length and that are at least 80%, at least 90% or 100% complementary to each other; or (2) one shRNA, which refers to a short hairpin ribonucleic acid that is single-stranded and that contains nucleotides that form a duplex region of 18-30 base pairs that are at least 80% complementary, at least 90% complementary or 100% complementary to each other, a stem region, a loop region and optionally an overhang region of up to six nucleotides in length. In an shRNA, within a duplex there is an antisense region and a sense region (that may also be referred to as strands, even though they are part of the same polynucleotide) that are each 18-30 nucleotides in length. Methods for using shRNA for inhibition of a target gene are well-known to persons of ordinary skill in the art and are described in U.S. 2010/0256222, published Oct. 7, 2012, the entire disclosure of which is incorporated by reference. Regardless of whether using two separate strands that form an siRNA or a single strand of shRNA, a region of the antisense strand that is 18-30 nucleotides in length is at least 80%, at least 90% or 100% complementary to a region of a target.

In any of the embodiments of the present invention, the compositions that are used may comprise a sequence referred to as an antisense sequence that is at least 80%, at least 90% or 100% complementary to a target sequence in Table I and a sense sequence that is at least 80%, is at least 90% or completely complementary to the antisense sequence. Throughout all of the tables, when a U is recited and unless otherwise specified, the sequence also includes instances in which a T is present instead of some of the Us or instead of all of the Us.

Optionally, siRNA molecules are introduced that contain one or more chemical modifications. Examples of chemical modifications that may be associated with the siRNA molecules include but are not limited to those recited in U.S. 2010/0197023, published Aug. 5, 2010, the entire disclosure of which is incorporated by reference. As persons of ordinary skill in the art will recognize, chemical modification of siRNA is often more easily accomplished when an siRNA is chemically synthesized than when the siRNA is generated within a cell. Accordingly, in some embodiments when a vector capable of generating an siRNA is introduced into a cell, the siRNA that is generated contains no chemical modifications, whereas in other embodiments, a molecule or two molecules that are capable of forming a duplex from one single strand or from two separate strands is introduced into a cell and either has chemical modifications or has no chemical modifications.

As persons of ordinary skill in the art will recognize, there are at least two different points in various embodiments in which reliance on RNAi may be advantageous. First, when making a stable cell line, it may be advantageous to introduce a lentiviral shRNA or other vector or construct capable of producing or being replicated to make molecules that can partake in RNAi. A single vector may code for one or both strands of an siRNA that is formed from two different strands. If it encodes for only one strand, then optionally there is a second vector or construct that encodes for the second strand or other means are provided to generate the second strand. If the resulting duplex is an shRNA, then a single lentiviral shRNA, vector or construct may be used to generate the duplex.

A second time occurs after the cells have been growing in suspension. At that point in the process, one may introduce an siRNA such as one that has Thermo Fisher Scientific Biosciences Inc.'s (formerly Dharmacon Inc.) commercial Accell modifications to the cells, which may be in the form of a suspension that was produced from the cell line. The term “Accell” refers to a preferred siRNA structure comprising the following. The sense strand is 19 nucleotides long and has: (1) 2′-O-methyl modifications on positions 1 and 2 (counting from the 5′ terminus); (2) 2′-O-methyl modifications on all Cs and Us; and (3) cholesterol conjugated to the 3′ terminus via a C5 linker. The antisense strand is 21 nucleotides in length, has a 5′ phosphate modification, contains a F modification on all Cs and Us, forms a two nucleotide overhang when paired with the sense strand, and contains phosphorthioate modifications between: (1) the two nucleotides of the overhang; and (2) between the 3′ most nucleotide of the duplexed region and the first nucleotide of the overhang. Preferably, the overhang is UU.

In addition, Accell molecules may contain mismatches at one, two or all of positions 6, 13, and 19 (counting from the 5′ end of the sense strand). Preferably, these mismatches are generated by replacing the sense nucleotide with an alternative base, e.g., the same base that is on the antisense strand. In this way, the antisense strand retains complete complementarity with the target molecule. For additional details on the Accell modifications, see U.S. 2009/0209626 A1, published Aug. 20, 2009, the entire disclosure of which is incorporated by reference.

The above described Accell modified siRNAs are provided merely by way of example. Other modified or unmodified siRNA may be added to the extract and may be in the form of two separate strands or a single polynucleotide that forms an shRNA.

According to a third embodiment, the present invention provides a method of making a cell-free extract. In this method, one establishes a stable cell line harboring at least one shRNA construct capable of expressing a double stranded oligonucleotide, wherein the double stranded oligonucleotide inhibits expression of a gene selected from the group consisting of: CCL19, GPR62, LILRB1, MAP3K14, MRPL14, OPN5, SCNN1A, SLC37A2 and TTYH3. By way of example, within a cell, a virally delivered shRNA may integrate into the cell's genome and be present there in one or more copies. In these embodiments, there may be a multiplicity of infection (“MOI”) of one or a MOI of up to 10 or a MOI of up to 50.

The inhibition may be complete or there may be at least 90% inhibition, at least 80% inhibition, at least 70% inhibition, at least 60% inhibition or at least 50% inhibition relative to the cell-line in the absence of the construct. From this cell line, one collects extract from the cell. The double stranded oligonucleotide may comprise a sequence that is identified by one or more rational design criteria such as those described in U.S. 2012/0052487, published Mar. 1, 2012, the entire disclosure of which is incorporated by reference. In this embodiment, the double stranded oligonucleotides, regardless of whether formed from one strand or from two strands, work by entering the RNAi machinery of the cell and directly or indirectly suppress the protein products.

According to a fourth embodiment, the present invention provides a method of making a cell-free extract comprising: (a) establishing a stable cell line harboring at least one shRNA construct capable of expressing a double stranded oligonucleotide, wherein the double stranded oligonucleotide comprises a sequence that is complementary to a region of a target gene selected from the group consisting of: CCL19, GPR62, LILRB1, MAP3K14, MRPL14, OPN5, SCNN1A, SLC37A2 and TTYH3; and (b) collecting extract from the cell. The double stranded oligonucleotide may directly silence or reduce expression of one or more of the aforementioned genes and/or indirectly affect expression by silencing or reducing expression of another gene, thereby either increasing or decreasing expression of one or more of the aforementioned genes. By way of a non-limiting example, when affecting translation levels indirectly, this may be due to off-target effects, and the gene on which the double stranded oligonucleotide works may, for example, be part of the same pathway.

Thus, in various embodiments, one or more siRNAs or shRNAs may be added to the cell-line before obtaining the extract and/or directly to the extract itself in order to induce a phenotype. In some of these embodiments, none, less than all or all of the siRNAs or shRNAs act through an miRNA-like mechanism and not by directly targeting an mRNA that codes for the ultimate protein whose expression level is sought to be altered. In some of these or other embodiments, none, less than all or all of the siRNAs or shRNAs act by directly targeting mRNA that is or is not the ultimate protein, the expression of which is sought to be reduced. Various embodiments of the present invention are directed to methods that implicate either or both of these pathways. Additionally, various embodiments of the present invention are directed to the individual siRNA or shRNA or to pools or kits that comprise two or more of them.

According to a fifth embodiment, the present invention provides a method of making a cell-free extract comprising introducing at least one miRNA mimic from the group consisting of mimics of hsa-miR-155, hsa-miR-1912, hsa-miR-200b, hsa-miR-200c, hsa-miR-219-2-3p, hsa-miR-299-3p, hsa-miR-451, hsa-miR-634, hsa-miR-877*, and hsa-miR-941 into a cell. The mimics may be introduced passively or they may be created in the cell through the introduction of a plasmid or other vector such as a Lentiviral SMARTvector (Thermo Fisher Scientific Biosciences Inc.) backbone, a transient mammalian expression vector, or another retroviral backbone such as an adeno-associated virus into the cell under appropriate conditions. The conditions may, for example, allow a virally-delivered shRNA-type construct to be integrated into the cell line at one or more copies, e.g., 1-50, 2-40, or 5-25 copies.

As persons of ordinary skill in the art know, an miRNA mimic is a synthetic or expressed RNA that acts as a functional equivalent to an endogenous human miRNA. Methods for expressing miRNA in vivo are well-known to persons of ordinary skill in the art and are disclosed in U.S. 2010/0292310, published Nov. 18, 2010, the entire disclosure of which is incorporated by reference.

As persons of ordinary skill in the art are also aware, miRNA may be in the form of small molecules, e.g., 17 to 25 nucleotides long. They can act by negatively regulating the expression of a gene that contains a sequence that is complementary to that of the miRNA. A given naturally occurring miRNA can regulate expression of tens to hundreds of genes. miRNA mimics, which may be non-naturally occurring oligonucleotides may be designed with sequences that in whole or in part are complementary to the sequences of one or more 3′UTR regions of one or more genes. Additionally, they may be unmodified or chemically modified and may be created or introduced as a 17-mer to 25-mer or as part of a duplex or within a scaffolding.

By way of non-limiting examples, the miRNA mimic may contain a mature sequence, e.g., as listed in Table III. In some embodiments, the mimic consists of, consists essentially of or comprises that sequence. In other embodiments, it may also contain a star sequence that is at least 80%, is at least 90% or is 100% complementary to the mature sequence. In some embodiments, the mature sequence and star sequence are housed in a scaffolding that is the same as or derived from a naturally occurring miRNA that is different from the miRNA from which the mature strand is derived. Preferably, the miRNA scaffold expresses and processes well inside the cell, and also preferentially loads the active strand into RISC, and thus, they may form a non-naturally occurring miRNA.

By way of a non-limiting example, a non-naturally occurring miR-196a-2 miRNA may comprise a stem-loop structure derived from miR-196a-2 (from any species, including homo sapiens) in which the stem of the stem-loop structure incorporates a mature strand-star strand duplex where the mature strand sequence is distinct from the endogenous mature strand sequence of miR-196a-2 and optionally distinct from the endogenous mature strand sequence of any miRNA. For example, it may be a scaffolding derived from a pre-miRNA or pri-miRNA structure.

Similarly, the mimic may be delivered as part of a non-naturally occurring miR-30 miRNA, which refers to a pre-miRNA or pri-miRNA comprising a miR-30 scaffold (i.e. a miRNA scaffold derived from miR-30) and a mature strand sequence that is not derived from miR-30 and optionally is distinct from the endogenous mature strand sequence of any miRNA. By way of further example, the mimic may be delivered as part of a non-naturally occurring miR-204 miRNA, which refers to a pre-miRNA or pri-miRNA comprising a miR-204 miRNA scaffold (i.e. a miRNA scaffold derived from miR-204) and a mature strand sequence that is not derived from miR-204 and optionally is distinct from the endogenous mature strand sequence of any miRNA.

The miRNA scaffold may also include additional 5′ and/or 3′ flanking sequences (for example, where it is desired to provide non-naturally occurring miRNA as a pri-miRNA that is first processed by Drosha to yield a pre-miRNA). Such flanking sequences flank the 5′ and/or 3′ ends of the stem-loop and range from about 5 nucleotides in length to about 600 nucleotides in length, preferably from about 5 nucleotides to about 150 nucleotides in length. The flanking sequences may be the same as the endogenous sequences that flank the 5′ end and/or the 3′ of the stem-loop structure of endogenous miRNA from which the miRNA scaffold is derived or they may be different by virtue of the addition, deletion, or substitution of one or more base pairs.

For example, a miR-196a-2 miRNA scaffold (and a non-naturally occurring miR-196a-2 miRNA obtained by cloning a mature strand sequence and a star strand sequence not from a naturally occurring miR-196a-2, thereunto) may include a 5′ and/or 3′ flanking sequence that is the same as the endogenous sequences that flank the 5′ end and/or the 3′ end of the stem-loop structure of endogenous miR-196a-2 miRNA.

As with the siRNA described above, the miRNA may be introduced to the cell prior to extraction, or to the extract following extraction, or at both times. Furthermore, different mimics may be introduced at the same or at different times.

According to another embodiment, the present invention provides a method of making a cell-free extract by reducing expression of a gene. In this method, one introduces an oligonucleotide into a cell, wherein the oligonucleotide reduces expression of a gene. An oligonucleotide is considered to be introduced into a cell when it is directly introduced in a form that may cause reduction of expression of gene, when it is generated from another molecule or molecules that are introduced into or already present in the cell (such as in the form of one or more vectors described in this specification or otherwise known to persons of ordinary skill in the art) or when one or more parts of the oligonucleotide are generated outside of the cell and then are processed or added to units within the cell. The oligonucleotide may, for example, be an siRNA formed from two separate strands, an shRNA or an miRNA mimic.

The oligonucleotide is selected and designed so that it can completely or partially reduce expression of a gene in a cell. For example, it may reduce expression by at least 10%, at least 20%, at least 30%, at least 40% , at least 50%, at least 60%, at least 70%, at least 80% or at least 90% relative to the expression level in the cell in the absence of the oligonucleotide. As persons of ordinary skill in the art will recognize, the ability of an oligonucleotide to reduce expression may in part or in whole be determined by its degree of complementarity to a target sequence and its concentration. In some embodiments, there is at least 80%, at least 90% or 100% complementarity of the oligonucleotide or when there are two strands, of one strand of the oligonucleotide and a region of the target. In some embodiments, when introducing unmodified siRNA, one may use the siRNA in a concentration of from 10 nM to 1 uM. For modified siRNA, such as those with the Accell modifications described in this application, in the absence of a delivery reagent the siRNA may be present in an amount of 500 nM to 10 uM. For vector-delivered si/miRNA, preferably the number of copies or the MOI is 1 to 50, 2-40 or 5-25.

In some embodiments, the gene that is targeted by the oligonucleotide negatively regulates translation. A gene is considered to regulate translation negatively if the presence of a product of the gene causes the rate or absolute amount of translation to decrease relative to the absence of the product. The product may, for example, be a protein or an RNA sequence. Furthermore, the negative regulation may be direct or indirect. For example, the product may be an inhibitor of an enzyme that participates in translation or the product may inhibit the activity of a first compound that is necessary to activate a second compound that is involved in translation.

Following introduction of the oligonucleotide, the cell is maintained under conditions that permit it to reduce expression of the gene, and then an extract is collected from the cell.

By way of a non-limiting example, according to one method of making a cell-free extract, one introduces at least one oligonucleotide comprising, consisting essentially of or consisting of at least one of SEQ ID NO: 37-48 and/or a complementary sequence into a cell and collects an extract from the cell. In other embodiments, combinations of oligonucleotides are introduced simultaneously or sequentially. By way of a non-limiting example, the combinations may be of one or more oligonucleotides, in the form of ssRNA, dsRNA or a part of a vector that comprises, consists essentially of or consists of sequence that correspond to the following sets of SEQ ID NOs, to complements of the following sets of SEQ ID NOs or both the following sets of SEQ ID NOs and the complements thereof: 37 and 38; 37 and 39; 37 and 40; 38 and 39; 38 and 40; 39 and 40; 37, 38 and 39; 37, 38 and 40; 37, 39 and 40; 38, 39 and 40; 37, 38, 39 and 40; 41 and 42; 41 and 43; 41 and 44; 42 and 43; 42 and 44; 43 and 44; 41, 42, and 43; 41,42 and 44; 41, 43 and 44; 42, 43 and 44; 41, 42, 43 and 44; 45 and 46; 45 and 47; 45 and 48;

46 and 47; 46 and 48; 47 and 48; 45, 46 and 47; 45, 46 and 48; 46, 47 and 48; and 45, 46, 47 and 48.

Various embodiments of the present invention call for obtaining an extract from a cell. Methods for obtaining an extract from a cell are well-known to persons of ordinary skill in the art. An example of this type of method is described in U.S. Pat. No. 8,012,712, issued Sep. 2, 2011, column 8, lines 23-67, which is incorporated by reference.

By way of non-limiting examples, the cells from which an extract is obtained may be from one of the following cell lines: HeLa S3, other HeLa cells, Huh7, CHO or HEK293. Furthermore, in some embodiments, the extract from these cells is supplemented with one or more translation initiation factors such as eIF2 (eukaryotic translation initiation factor 2), eIF2B (eukaryotic translation initiation factor 2B) or eIF4E (eukaryotic translation initiation factor 4E) and/or a translational regulator, e.g., p97 (a homologue to the C-terminal two methods of eIF4G). For example, both eIF2 and eIF2B may be supplemented, or eIF2, eIF2B and eIF4G may be supplemented, or p97, eIF2, and eIF4G may be supplemented.

Each of the aforementioned mimics, the miRbase ID numbers for which are provided in Table III may be introduced in combination with one or more siRNAs that target one or more genes identified in Table I. Within Table I, four target sequences are provided for each gene. By combining the siRNA and mimics, one may be able to generate modified stable cell lines that produce desirable cell-free extracts. Table IV provides combinations of single mimics and siRNAs for single targets. As a person of ordinary skill in the art will appreciate, pools of two or more mimics can be combined with siRNA for one or more targets, and pools of two or more siRNAs can be combined with one or more mimics.

Table II provides an additional group of targets and four target sequences for each gene. The sequences in Table II (SEQ ID NOs: 37-48) may be part of a duplex such as described elsewhere in this application. These sequences have the ability to act as miRNA mimics and thus they may be used for the same purposes for which miRNA mimics are used. In any one application, one or more of the recited sequences may be used. When a combination of the sequences are used, a person of ordinary skill in the art may select to use two or more that are associated with the same target in the table or two or more that are associated with different targets. Additionally or alternatively, they may be used in combination with other oligonucleotides that are recited in this disclosure, e.g., (1) one or more oligonucleotides comprising, consisting essentially of or consisting of SEQ ID NOs: 1-36, the complement thereof or both SEQ ID NOs: 1-36 and the complement thereof; (2) one or more oligonucleotides comprising, consisting essentially of or consisting of SEQ ID NOs: 49-58, the complement thereof or both SEQ ID NOs: 49-58, and the complement thereof; or (3) both (i) one or more oligonucleotides comprising, consisting essentially of or consisting of SEQ ID NOs: 1-36, the complement thereof or both SEQ ID NOs: 1-36 and the complement thereof; and (ii) one or more oligonucleotides comprising, consisting essentially of or consisting of SEQ ID NOs: 49-58, the complement thereof or both SEQ ID NOs: 49-58, and the complement thereof. As persons of ordinary skill in the art will recognize, the aforementioned degrees of complementarity may be at least 80%, at least 90% or 100%.

By way of non-limiting examples, the cell-free extracts of the present invention may be used in one or more of the following applications: (i) experiments to characterize protein-activity; (ii) experiments to characterize protein-protein interactions and protein-nucleic acid interactions; (iii) rapid and high-throughput expression of mutant truncated proteins for functional analysis; (iv) expression of mammalian proteins with proper glycosylation and native post-translational modifications (PTM); (v) labeling of proteins with stable structural analysis; (vi) production of functional virions or toxic polypeptides; (vii) analysis of components required for protein folding, protein stability or protein degradation; and (viii) production of proteins with incorporation of non-natural amino acids (i.e., isotype-labeled, fluorescently-labeled, azide-labeled, etc.).

Advantages of protein expression using the various embodiments of the present invention include but are not limited increase efficiency when expressing large, hard-to-fold proteins and proteins that require some level of mammalian-specific glycosylation. Examples of the aforementioned types of proteins include but are not limited to: Rb1 (106 kD), gp120 (120 kD), GCN2 (160 kD), Dicer (200kD), MTOR (260 kD), and hCG (human chorionic gonadotropin).

In some embodiments, when using these cell-free lysates, mRNA for translation is present in an amount between 0.001 mg/ml and 2 mg/ml, or between 0.01 mg/ml and 1 mg/ml, or between 0.05 mg/ml and 0.5 mg/ml.

By using the extracts of the present invention, one can increase the optimization of a cell-free expression system, including the production of proper post-translational modifications. The cell-free extracts may translate proteins directly from mRNA or first transcribe from DNA to mRNA and then translate the mRNA into proteins. When starting with DNA, one may couple transcription and translation in the same environment, e.g., in the same vial, or one may link them, in which case, DNA is transcribed to mRNA in one environment, and all or part of that environment is then combined with the cell-free extract for translation. If transcription is to occur within the extract, then preferably the necessary enzymes and associated other components for transcription are also present, e.g., free ribonucleotides for incorporation into a growing mRNA strand.

By way of a non-limiting example, in a one-step coupled reaction, one may use 0.1 μg to 2.0 μg (e.g., 1 μg) of plasmid DNA in a 20-30 μl (e.g., 25 μl) lysate reaction or 5-50 μl/ml (e.g., 40 μg/ml). More than one plasmid DNA can be added to a given reaction to produce more than one protein in the coupled reaction. This allows two or more proteins to be studied in the same reaction, such as multi-subunit protein complexes or signaling pathways.

As persons of ordinary skill in the art will recognize, there are advantages to be realized by adding: (i) a plurality of different siRNA, e.g., an siRNA or an shRNA; or (ii) at least one siRNA or shRNA and an miR mimic. The effect of the combination can be additive, subtractive or synergistic. Furthermore, by adding a plurality of these types of oligonucleotides to the cell prior to extraction and incubation or after extraction, a person of ordinary skill in the art can control the expression of target products.

In another embodiment, the present invention provides a kit that comprises a human cell line lysate that produces functional protein within less than 90 minutes. Optionally, the kit is an mRNA kit and comprises: (1) an active lysate that has one or more if not all of ribosomes, tRNAs, aminoacyl-tRNA synthetases, protein factures, GTP, ATP, Mg²⁺ and K⁺; (2) an energy mix; (3) a salt solution; (4) an RNase inhibitor; and (5) additional proteins and amino acids.

When using the mRNA kit, one may, for example, combine approximately 1 microgram of an mRNA template with the components of the kit under conditions that are conducive to translation, e.g., 1-3 hours, at 28-30° C. As persons of ordinary skill in the art will recognize, a temperature of approximately 28° C. is conducive to glycosylation. If one begins with a circular or linear DNA template, one will first need to transcribe it into mRNA. By way of a non-limiting example, one may conduct a transcription reaction at approximately 32° C. for one hour in order to generate approximately 2-3 micro liters of product and then combine this product with the compounds of an mRNA kit. Thus, a DNA kit may contain all of the components of an mRNA kit and additional components that allow for translation.

Various aspects of the present invention have been described for use in connection with one or more embodiments. However, unless explicitly stated or apparent from context, each feature described above in any one embodiment may be used in connection with any and all embodiments.

TABLE I
siGenome Targets

| | | Entrez | | | |
|---|---|---|---|---|---|
| | | Gene | Target | SEQ ID | |
| Gene Symbol | Gene Name | ID | Accession | NO: | Target Sequences |
| CCL19 | Ligand chemokine | 6363 | NM_006274 | 1 | CUGGGUACAUCGUG |
| | C-C motif 19 | | | | AGGAA |
| | | | | 2 | CUGCAGAGGACCUC |
| | | | | | AGCCA |
| | | | | 3 | CUGCAGGGUGCCUG |
| | | | | | CUGUA |
| | | | | 4 | GAACUUCCACUACC |
| | | | | | UUCUC |
| GPR62 | G protein-coupled | 118442 | NM_080865 | 5 | GGACAAAGCUACUG |
| | receptor 62 | | | | AAACU |
| | | | | 6 | GACCUCAGCUGCAC |
| | | | | | CCAUU |
| | | | | 7 | CCUAAGGGCUCACA |
| | | | | | ACCAA |
| | | | | 8 | GCCCACAACACCAG |
| | | | | | UAUUU |
| LILRB1 | Leukocyte | 10859 | NM_001081637 | 9 | UCACAGAGCUCCAA |
| | immunoglobulin- | | | | ACCCU |
| | like receptor, | | | 10 | CGGUAUCGCUGUUA |
| | subfamily B, | | | | CUAUG |
| | member 1 | | | 11 | GAUCAACGUACCAA |
| | | | | | UCUCA |
| | | | | 12 | GCACACACAGCCUG |
| | | | | | AGGAU |
| MAP3K14 | Mitogen-activated | 9020 | NM_003954 | 13 | UCUCAAAGCUCGCG |
| | protein kinase | | | | GGACA |
| | kinase kinase 14 | | | 14 | GGGAAAGCGUCGCA |
| | | | | | GCAAA |
| | | | | 15 | CGCCAAAUCAAGCC |
| | | | | | AAUUA |
| | | | | 16 | GAUCCUGAAUGACG |
| | | | | | UGAUU |
| MRPL14 | Mitochondrial | 64928 | NM_032111 | 17 | CAACAACGUGGUCC |
| | ribosomal protein | | | | UCAUU |
| | L14, nuclear gene | | | 18 | GCUCCUCGCUGCAU |
| | encoding | | | | CCAUG |
| | mitochondrial | | | 19 | GGGAACAGCCCAUA |
| | protein | | | | CCAUC |
| | | | | 20 | CAGAAGAUGACGCG |
| | | | | | GGUAC |
| OPN5 | Opsin 5, transcript | 221391 | NM_001030051 | 21 | CCAAAGAAGUAGCU |
| | variant 2 | | | | CAUUU |
| | | | | 22 | UGACAAAGGUAGCG |
| | | | | | AUGUU |
| | | | | 23 | ACUUAAAGCUCCUC |
| | | | | | GGGAU |
| | | | | 24 | AGAUCAUUGCCAAG |
| | | | | | GUUAA |
| SCNN1A | sodium channel, | 6337 | NM_001038 | 25 | UCAAGGAGCUGAAC |
| | nonvoltage-gated | | | | UACAA |
| | 1 alpha subunit, | | | 26 | GCAGUGAUGUUCCU |
| | transcript variant 1 | | | | GUUGA |
| | | | | 27 | GGGUAAUGGUGCAC |
| | | | | | GGGCA |
| | | | | 28 | CCUACAGGUACCCG |
| | | | | | GAAAU |
| SLC37A2 | solute carrier | 219855 | NM_198277 | 29 | CUGCUGACCUUCCU |
| | family 37 | | | | AAUUU |
| | (glycerol-3- | | | 30 | GGACAACGCCUUCC |
| | phosphate | | | | UCAUC |
| | transporter), | | | 31 | UUGCCAAGCUGGUC |
| | member 2, | | | | AGUUA |
| | transcript variant 1 | | | 32 | GCAUCUGGGUGAAC |
| | | | | | GGGCA |
| TTYH3 | tweety homolog 3 | 80727 | NM_025250 | 33 | CCGCACACCUGGCA |
| | | | | | GCAAA |
| | | | | 34 | GCAGUGGGAUUCUA |
| | | | | | CGGCA |
| | | | | 35 | CCAGAACGCUAAUU |
| | | | | | UCCAG |
| | | | | 36 | CGGAGCAGGUGGAU |
| | | | | | CUCUA |

TABLE II
Targets and Sequences that behave like miRNA Mimics

| Gene | | Entrez | Target | SEQ ID | |
|---|---|---|---|---|---|
| Symbol | Gene Name | Gene ID | Accession | NO: | Target Sequences |
| PP1R14C | protein | 81706 | NM_030949 | 37 | GAGCUGCUUUCUCGGAUAA |
| | phosphatase | | | 38 | UGCCAGAGGUAGAAAUUGA |
| | 1, regulatory | | | 39 | CUACAAACCAACAGAGGAA |
| | (inhibitor) | | | 40 | CCGCAGAAGAAGAGUGUAU |
| | subunit 14C | | | | |
| GNRH1 | gonadotropin- | 2796 | NM_000825; | 41 | GAAAGAGAGAUGCCGAAAA |
| | releasing | | NM_001083111 | 42 | AGUCAAAGAGGUUGGUCAA |
| | hormone 1 | | | 43 | UGGCAGAAACCCAACGCUU |
| | (luteinizing- | | | 44 | AAGUCUGAUUGAAGAGGAA |
| | releasing | | | | |
| | hormone) | | | | |
| KCNJ4 | potassium | 3761 | NM_004981; | 45 | AGAACGAGCUGGCCCUUAU |
| | inwardly- | | NM_152868 | 46 | CAACGUGGGCUAUGACAUC |
| | rectifying | | | 47 | GGCCUCCUCUUCUGGUGUA |
| | channel, | | | 48 | GCAACAAGUCGCAGCGCUA |
| | subfamily J, | | | | |
| | member 4 | | | | |

TABLE III
miRNA Mimics

| | | | SEQ |
|---|---|---|---|
| | | | ID |
| miRNA Symbol | miRbase ID | Mature miRNA Sequence | NO: |
| hsa-miR-155 | MI0000681 | UUAAUGCUAAUCGUGAUAGGGGU | 49 |
| hsa-miR-1912 | MI0008333 | UACCCAGAGCAUGCAGUGUGAA | 50 |
| hsa-miR-200b | MI0000342 | UAAUACUGCCUGGUAAUGAUGA | 51 |
| hsa-miR-200c | MI0000650 | UAAUACUGCCGGGUAAUGAUGGA | 52 |
| hsa-miR-219- | MI0000740 | AGAAUUGUGGCUGGACAUCUGU | 53 |
| 2-3p | | | |
| hsa-miR-299- | MI0000744 | UAUGUGGGAUGGUAAACCGCUU | 54 |
| 3p | | | |
| hsa-miR-451 | MI0001729 | AAACCGUUACCAUUACUGAGUU | 55 |
| hsa-miR-634 | MI0003649 | AACCAGCACCCCAACUUUGGAC | 56 |
| hsa-miR-877* | MI0005561 | UCCUCUUCUCCCUCCUCCCAG | 57 |
| hsa-miR-941 | MI0005763 | CACCCGGCUGUGUGCACAUGUGC | 58 |

TABLE IV
Examples of Combinations of Inhibitors and Mimics

| miRNA Mimic | siRNA target |
|---|---|
| hsa-miR-155 | CCL19 |
| hsa-miR-155 | GPR62 |
| hsa-miR-155 | LILRB1 |
| hsa-miR-155 | MAP3K14 |
| hsa-miR-155 | MRPL14 |
| hsa-miR-155 | OPN5 |
| hsa-miR-155 | SCNN1A |
| hsa-miR-155 | SLC37A2 |
| hsa-miR-155 | TTYH3 |
| hsa-miR-1912 | CCL19 |
| hsa-miR-1912 | GPR62 |
| hsa-miR-1912 | LILRB1 |
| hsa-miR-1912 | MAP3K14 |
| hsa-miR-1912 | MRPL14 |
| hsa-miR-1912 | OPN5 |
| hsa-miR-1912 | SCNN1A |
| hsa-miR-1912 | SLC37A2 |
| hsa-miR-1912 | TTYH3 |
| hsa-miR-200b | CCL19 |
| hsa-miR-200b | GPR62 |
| hsa-miR-200b | LILRB1 |
| hsa-miR-200b | MAP3K14 |
| hsa-miR-200b | MRPL14 |
| hsa-miR-200b | OPN5 |
| hsa-miR-200b | SCNN1A |
| hsa-miR-200b | SLC37A2 |
| hsa-miR-200b | TTYH3 |
| hsa-miR-200c | CCL19 |
| hsa-miR-200c | GPR62 |
| hsa-miR-200c | LILRB1 |
| hsa-miR-200c | MAP3K14 |
| hsa-miR-200c | MRPL14 |
| hsa-miR-200c | OPN5 |
| hsa-miR-200c | SCNN1A |
| hsa-miR-200c | SLC37A2 |
| hsa-miR-200c | TTYH3 |
| hsa-miR-219-2-3p | CCL19 |
| hsa-miR-219-2-3p | GPR62 |
| hsa-miR-219-2-3p | LILRB1 |
| hsa-miR-219-2-3p | MAP3K14 |
| hsa-miR-219-2-3p | MRPL14 |
| hsa-miR-219-2-3p | OPN5 |
| hsa-miR-219-2-3p | SCNN1A |
| hsa-miR-219-2-3p | SLC37A2 |
| hsa-miR-219-2-3p | TTYH3 |
| hsa-miR-299-3p | CCL19 |
| hsa-miR-299-3p | GPR62 |
| hsa-miR-299-3p | LILRB1 |
| hsa-miR-299-3p | MAP3K14 |
| hsa-miR-299-3p | MRPL14 |
| hsa-miR-299-3p | OPN5 |
| hsa-miR-299-3p | SCNN1A |
| hsa-miR-299-3p | SLC37A2 |
| hsa-miR-299-3p | TTYH3 |
| hsa-miR-451 | CCL19 |
| hsa-miR-451 | GPR62 |
| hsa-miR-451 | LILRB1 |
| hsa-miR-451 | MAP3K14 |
| hsa-miR-451 | MRPL14 |
| hsa-miR-451 | OPN5 |
| hsa-miR-451 | SCNN1A |
| hsa-miR-451 | SLC37A2 |
| hsa-miR-451 | TTYH3 |
| hsa-miR-634 | CCL19 |
| hsa-miR-634 | GPR62 |
| hsa-miR-634 | LILRB1 |
| hsa-miR-634 | MAP3K14 |
| hsa-miR-634 | MRPL14 |
| hsa-miR-634 | OPN5 |
| hsa-miR-634 | SCNN1A |
| hsa-miR-634 | SLC37A2 |
| hsa-miR-634 | TTYH3 |
| hsa-miR-877* | CCL19 |
| hsa-miR-877* | GPR62 |
| hsa-miR-877* | LILRB1 |
| hsa-miR-877* | MAP3K14 |
| hsa-miR-877* | MRPL14 |
| hsa-miR-877* | OPN5 |
| hsa-miR-877* | SCNN1A |
| hsa-miR-877* | SLC37A2 |
| hsa-miR-877* | TTYH3 |
| hsa-miR-941 | CCL19 |
| hsa-miR-941 | GPR62 |
| hsa-miR-941 | LILRB1 |
| hsa-miR-941 | MAP3K14 |
| hsa-miR-941 | MRPL14 |
| hsa-miR-941 | OPN5 |
| hsa-miR-941 | SCNN1A |
| hsa-miR-941 | SLC37A2 |
| hsa-miR-941 | TTYH3 |

### EXAMPLES

### Example 1

HeLaS3 cells stably expressing Gaussia Luciferase under a TK (Thymidine Kinase) promoter were used in a wet reverse transfection at a density of 20,000 cells per well, originating from an expanded bank of cells initially frozen at the same passage number. The Thermo Fisher Scientific Biosciences Inc. siGENOME SMARTpool (18,164 gene targets) and miRIDIAN miRNA Mimic (miRbase 13.0, 869 Mimics) libraries were reverse transfected in triplicate using a JANUS workstation (PerkinElmer) and Multidrop Combi (Thermo) Reagent Dispenser (50 nM final concentration, 120 uL final volume using 0.3 uL/well of DharmaFECT 1 reagent in 96-well tissue culture plates).

After transfection, cells were incubated for 72 hours (37 degrees C., 5% CO₂). Upon media aspiration, wells were washed once with PBS (100 μL), treated with fresh media, and allowed to incubate for an additional 2 hours. In a separate white bottom assay plate (Corning Costar), for each well, 20 μL of cell media was then mixed with 20 μL of prepared Thermo Scientific Pierce Gaussia Luciferase Flash assay reagent, shaken for 30 seconds, and incubated for 3 minutes at room temperature.

Enhanced luminescence was read on an EnVision Multilabel Reader (PerkinElmer). Each plate was subjected to quality control of the z′ factor calculated between the eight non-targeting control (NTC) wells and the eight down regulation positive control wells (SMARTpool targeting EIF2B2) being above zero. For hit selection, a robust z score was calculated for each well using the median signal of the NTC wells (instead of the median of all samples). From the primary screen data, 110 hits with increased secreted Gaussia expression (robust z score >1.3) were selected for confirmation. Those 110 hits were subjected to the same assay two more times through rescreening or confirmation of the results. From those 110 hits, the top nine scoring targets were selected for inclusion in Table I.

### Example 2

One of the top gene targets in a primary siRNA screen was SCNN1A, which is the major subunit of the epithelial sodium channel, or ENaC. A search for the top gene hit targets in DrugBank (www.drugbank.ca) revealed that SCNN1A is the target of a known antihypertensive drug, amiloride (3,5-diamino-6-chloro-N-(diaminomethylene) pyrazine 2-carboxamide). A literature search revealed connections between amiloride treatment of cells and effects on stress pathways, specifically the endoplasmic reticulum stress pathway. Because stress pathways in cells are the major source of phosphorylation of eIF2-alpha, which inhibits translational capacity, the inventors investigated whether treatment of cells with amiloride in culture would have any effect on translational capacity, with or without added cell stressors.

HeLaS3-Gluc cells were plated at 10K/well and incubated overnight. Subsequently, they were pre-treated with amiloride and incubated for 1 hour. Amiloride was used at increasing doses, both alone and in the presence of cellular stressors tunicamycin or thapsigargin. When the ER stressors were included, incubation was for 2 hours, at which time Gluc was measured. The media was then changed, and amiloride and ER stressors were added again. This time incubation was overnight. The media was again changed and the amount of Gluc made was measured after two hours. Amiloride was used in amounts of 0-500 μM. Tunicamycin was used in an amount of 5 μM. Thapsigargin was used in an amount of 2 μM. While treatment of cells with amiloride alone in the absence of stress did not lead to increased luciferase output (in contrast to the effect of SCNN1A knockdown), the reduced luciferase output as a function of treatment with cellular stressors was rescued by treatment with an increasing amount of amiloride.

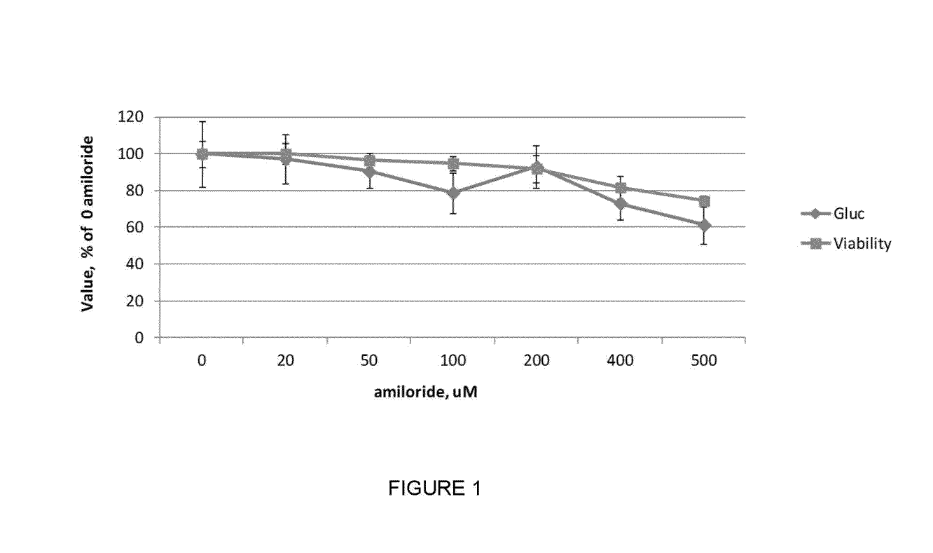

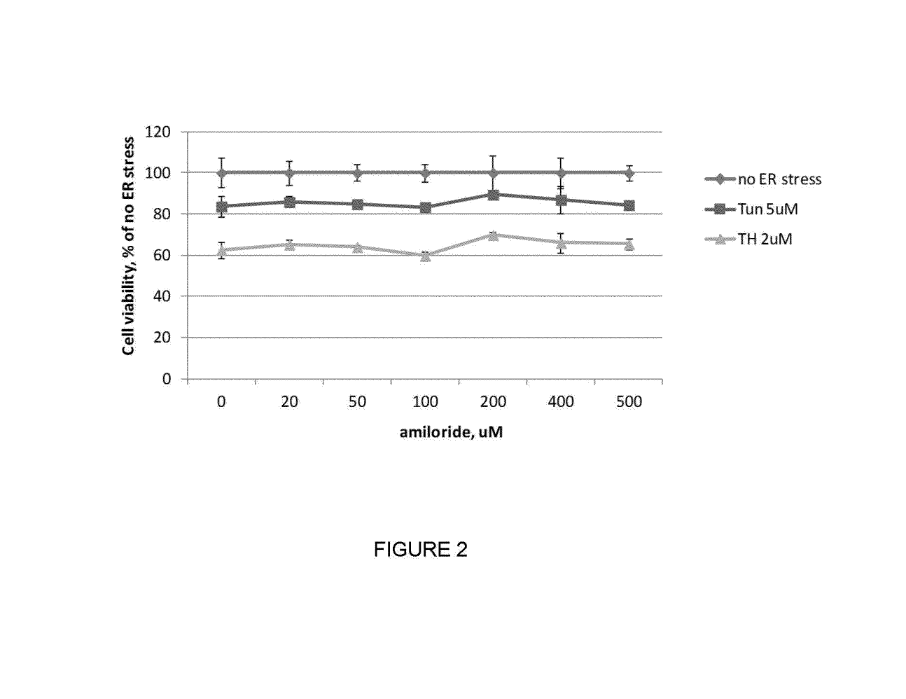

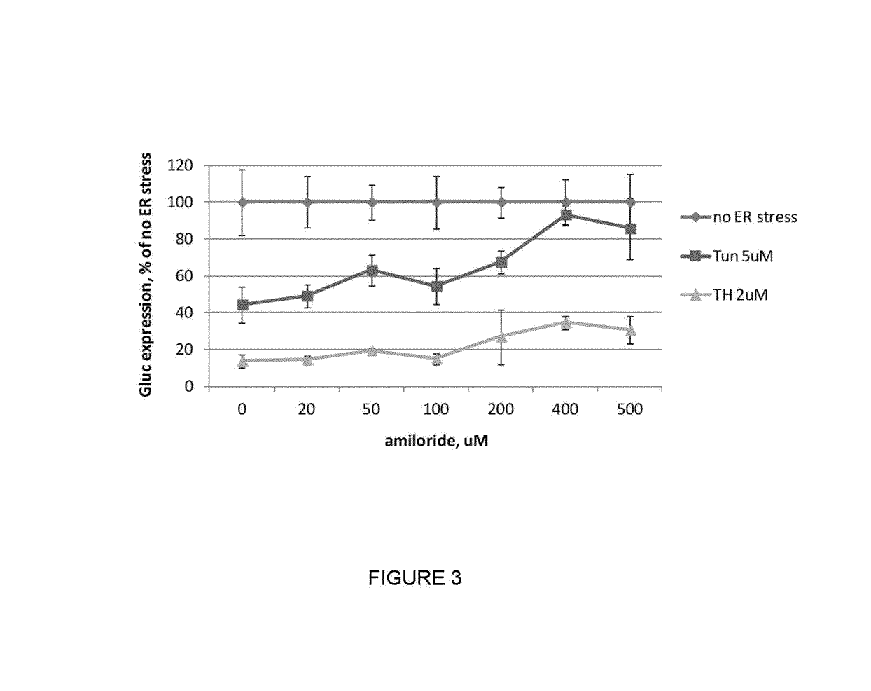

FIG. 1 illustrates the measurement of Gluc expression by, and the viability of, unstressed cells in the presence of amiloride. As the figure shows, increased amiloride leads to a decrease in viability and glucose expression. FIG. 2 illustrates the effect of ER stress on viability. As the figure also shows, increasing the amount of amiloride does not have an additional effect on cell viability in the presence of ER stress. However, as FIG. 3 shows, high doses of amiloride can restore Gluc expression that was reduced due to ER stress.

This suggests that under stress conditions, as occurs with the lysing of cells during the production of cell-free translation extract, addition of amiloride to the cells before lysis has an effect on the ultimate translational capacity of this extract, either alone or in combination with another cellular treatment, such as knockdown of one of the genes identified in the primary siRNA screen.

We claim:

1. An extract prepared from cells in which expression of a gene is inhibited, wherein the gene is selected from the group consisting of: CCL19, GPR62, LILRB1, MAP3K14, MRPL14, OPN5, SCNN1A, SLC37A2 and TTYH3.

2. The extract of claim 1, wherein the cells comprise at least one RNAi agent and the extract comprises a product of the gene in an amount of less than 50% of the product in an extract from cells that have an absence of the RNAi agent.

3. The extract of claim 2, wherein the product inhibits transcription of a nucleotide sequence, inhibits translation of a nucleotide sequence, inhibits transport of a protein or a polynucleotide or gene, or inhibits secretion of a protein or a polynucleotide or combination thereof.

4. The extract of claim 3, wherein the RNAi agent comprises a sequence selected from the group consisting of SEQ ID NO: 1-36 or a complement thereof.

5. The extract of claim 4, wherein the RNAi agent is an siRNA that has Accell modifications.

6. A method of making a cell-free extract comprising: (a) establishing a stable cell line harboring at least one shRNA construct capable of expressing a double stranded oligonucleotide, wherein the double stranded oligonucleotide inhibits expression of a gene selected from the group consisting of: CCL19, GPR62, LILRB1, MAP3K14, MRPL14, OPN5, SCNN1A, SLC37A2 and TTYH3; and (b) collecting an extract from the cell.

7. The method of claim 6, wherein the double stranded oligonucleotide comprises a sequence that is complementary to a region of the gene.

8. The method of claim 7, wherein the region of the gene is a sequence selected from the group consisting of SEQ ID NO: 1-36.

9. The method of claim 6, wherein the gene is a first gene and the double stranded oligonucleotide comprises a sequence that is complementary to a region of a second gene.

10. A method of making a cell-free extract comprising introducing at least one miRNA mimic from the group consisting of mimics of hsa-miR-155, hsa-miR-1912, hsa-miR-200b, hsa-miR-200c, hsa-miR-219-2-3p, hsa-miR-299-3p, hsa-miR-451, hsa-miR-634, hsa-miR-877, and hsa-miR-941 into a cell and collecting an extract from the cell.

11. The method claim 10, wherein the miRNA mimic is introduced within a scaffolding of hsa-miR-196a-2 or miR-204 or hsa-miR-30.

12. The method of claim 10, wherein the cell is HeLa S3.

13. A method of making a cell-free extract comprising:
(a) introducing an oligonucleotide into a cell, wherein said oligonucleotide reduces expression of a gene and wherein a product of said gene negatively regulates translation; and
(b) collecting an extract from said cell.

14. The method according to claim 13, wherein the oligonucleotide reduces expression of the gene by at least 50%.

15. The method according to claim 14, wherein the oligonucleotide is generated from a vector within the cell.

16. The method according to claim 15, wherein the oligonucleotide is an miRNA mimic.

17. The method according to claim 15, wherein the oligonucleotide is an siRNA that is formed from two separate strands or an shRNA.

18. The method according to claim 13, wherein the oligonucleotide comprises at least one of SEQ ID NO: 1-58 or a complement thereof.

19. The method according to claim 13, wherein the oligonucleotide is a first oligonucleotide and comprises at least one of SEQ ID NO: 1-36 or a complement thereof, and the method further comprises introducing into the cell, a second oligonucleotide that comprises at least one of SEQ ID NO: 37-48 or a complement thereof.

20. The method according to claim 13, wherein the oligonucleotide is a first oligonucleotide and comprises at least one of SEQ ID NO: 1-36 or a complement thereof, and the method further comprises introducing into the cell, a second oligonucleotide that comprises at least one of SEQ ID NO: 49-58. or a complement thereof.


SEQUENCE LISTING (58 sequences)

SEQ ID NO: 1 - RNA, 19 nt, Homo sapiens
cuggguacaucgugaggaa

SEQ ID NO: 2 - RNA, 19 nt, homo sapiens
cugcagaggaccucagcca

SEQ ID NO: 3 - RNA, 19 nt, Homo sapiens
cugcagggugccugcugua

SEQ ID NO: 4 - RNA, 19 nt, Homo sapiens
gaacuuccacuaccuucuc

SEQ ID NO: 5 - RNA, 19 nt, Homo sapiens
ggacaaagcuacugaaacu

SEQ ID NO: 6 - RNA, 19 nt, Homo sapiens
gaccucagcugcacccauu

SEQ ID NO: 7 - RNA, 19 nt, Homo sapiens
ccuaagggcucacaaccaa

SEQ ID NO: 8 - RNA, 19 nt, Homo sapiens
gcccacaacaccaguauuu

SEQ ID NO: 9 - RNA, 19 nt, Homo sapiens
ucacagagcuccaaacccu

SEQ ID NO: 10 - RNA, 19 nt, Homo sapiens
cgguaucgcuguuacuaug

SEQ ID NO: 11 - RNA, 19 nt, Homo sapiens
gaucaacguaccaaucuca

SEQ ID NO: 12 - RNA, 19 nt, Homo sapiens
gcacacacagccugaggau

SEQ ID NO: 13 - RNA, 19 nt, Homo sapiens
ucucaaagcucgcgggaca

SEQ ID NO: 14 - RNA, 19 nt, Homo sapiens
gggaaagcgucgcagcaaa

SEQ ID NO: 15 - RNA, 19 nt, Homo sapiens
cgccaaaucaagccaauua

SEQ ID NO: 16 - RNA, 19 nt, Homo sapiens
cagaagaugacgcggguac

SEQ ID NO: 17 - RNA, 19 nt, Homo sapiens
caacaacgugguccucauu

SEQ ID NO: 18 - RNA, 19 nt, Homo sapiens
gcuccucgcugcauccaug

SEQ ID NO: 19 - RNA, 19 nt, Homo sapiens
gggaacagcccauaccauc

SEQ ID NO: 20 - RNA, 19 nt, Homo sapiens
cagaagaugacgcggguac

SEQ ID NO: 21 - RNA, 19 nt, Homo sapiens
ccaaagaaguagcucauuu

SEQ ID NO: 22 - RNA, 19 nt, Homo sapiens
ugacaaagguagcgauguu

SEQ ID NO: 23 - RNA, 19 nt, Homo sapiens
acuuaaagcuccucgggau

SEQ ID NO: 24 - RNA, 19 nt, Homo sapiens
agaucauugccaagguuaa

SEQ ID NO: 25 - RNA, 19 nt, Homo sapiens
ucaaggagcugaacuacaa

SEQ ID NO: 26 - RNA, 19 nt, Homo sapiens
gcagugauguuccuguuga

SEQ ID NO: 27 - RNA, 19 nt, Homo sapiens
ggguaauggugcacgggca

SEQ ID NO: 28 - RNA, 19 nt, Homo sapiens
ccuacagguacccggaaau

SEQ ID NO: 29 - RNA, 19 nt, Homo sapiens
cugcugaccuuccuaauuu

SEQ ID NO: 30 - RNA, 19 nt, Homo sapiens
ggacaacgccuuccucauc

SEQ ID NO: 31 - RNA, 19 nt, Homo sapiens
uugccaagcuggucaguua

SEQ ID NO: 32 - RNA, 19 nt, Homo sapiens
gcaucugggugaacgggca

SEQ ID NO: 33 - RNA, 19 nt, Homo sapiens
ccgcacaccuggcagcaaa

SEQ ID NO: 34 - RNA, 19 nt, Homo sapiens
gcagugggauucuacggca

SEQ ID NO: 35 - RNA, 19 nt, Homo sapiens
ccagaacgcuaauuuccag

SEQ ID NO: 36 - RNA, 19 nt, Homo sapiens
cggagcagguggaucucua

SEQ ID NO: 37 - RNA, 19 nt, Homo sapiens
gagcugcuuucucggauaa

SEQ ID NO: 38 - RNA, 19 nt, Homo sapiens
ugccagagguagaaauuga

SEQ ID NO: 39 - RNA, 19 nt, Homo sapiens
cuacaaaccaacagaggaa

SEQ ID NO: 40 - RNA, 19 nt, Homo sapiens
ccgcagaagaagaguguau

SEQ ID NO: 41 - RNA, 19 nt, Homo sapiens
gaaagagagaugccgaaaa

SEQ ID NO: 42 - RNA, 19 nt, Homo sapiens
agucaaagagguuggucaa

SEQ ID NO: 43 - RNA, 19 nt, Homo sapiens
uggcagaaacccaacgcuu

SEQ ID NO: 44 - RNA, 19 nt, Homo sapiens
aagucugauugaagaggaa

SEQ ID NO: 45 - RNA, 19 nt, Homo sapiens
agaacgagcuggcccuuau

SEQ ID NO: 46 - RNA, 19 nt, Homo sapiens
caacgugggcuaugacauc

SEQ ID NO: 47 - RNA, 19 nt, Homo sapiens
ggccuccucuucuggugua

SEQ ID NO: 48 - RNA, 19 nt, Homo sapiens
gcaacaagucgcagcgcua

SEQ ID NO: 49 - RNA, 23 nt, Homo sapiens
uuaaugcuaaucgugauaggggu

SEQ ID NO: 50 - RNA, 22 nt, Homo sapiens
uacccagagcaugcagugugaa

SEQ ID NO: 51 - RNA, 22 nt, Homo sapiens
uaauacugccugguaaugauga

SEQ ID NO: 52 - RNA, 23 nt, Homo sapiens
uaauacugccggguaaugaugga

SEQ ID NO: 53 - RNA, 22 nt, Homo sapiens
agaauuguggcuggacaucugu

SEQ ID NO: 54 - RNA, 22 nt, Homo sapiens
uaugugggaugguaaaccgcuu

SEQ ID NO: 55 - RNA, 22 nt, Homo sapiens
aaaccguuaccauuacugaguu

SEQ ID NO: 56 - RNA, 22 nt, Homo sapiens
aaccagcaccccaacuuuggac

SEQ ID NO: 57 - RNA, 21 nt, Homo sapiens
uccucuucucccuccucccag

SEQ ID NO: 58 - RNA, 23 nt, Homo sapiens
cacccggcugugugcacaugugc